(19) United States
(12) Patent Application Publication
(10) Pub. No.: US 2019/0060368 A1
(43) Pub. Date: Feb. 28, 2019

(54) Mesenchymal Stem/Stromal Cell-Derived Extracellular Vesicles And Uses Thereof In Autoimmune Diseases

(71) Applicants: Ryang Hwa LEE, College Station, TX (US); Joo Youn OH, Seoul (KR); Darwin J. PROCKOP, College Station, TX (US); Dong-ki KIM, College Station, TX (US); Taeko Shigemoto KURODA, College Station, TX (US)

(72) Inventors: Ryang Hwa LEE, College Station, TX (US); Joo Youn OH, Seoul (KR); Darwin J. PROCKOP, College Station, TX (US); Dong-ki KIM, College Station, TX (US); Taeko Shigemoto KURODA, College Station, TX (US)

(73) Assignee: THE TEXAS A&M UNIVERSITY SYSTEM, College Station, TX (US)

(21) Appl. No.: 16/112,282

(22) Filed: Aug. 24, 2018

Related U.S. Application Data
(60) Provisional application No. 62/549,892, filed on Aug. 24, 2017.

(51) Int. Cl. A61K 35/28; A61K 9/08; A61P 37/06

(52) U.S. Cl. CPC A61K 35/28; A61P 37/06; A61K 9/08

(57) ABSTRACT

Pharmaceutically acceptable preparations of extracellular vesicles derived from activated MSCs are provided. These preparations are essentially free of MSCs, and demonstrate anti-inflammatory inhibiting pharmacological activity in vivo. Methods for using the preparations to prevent the onset of autoimmune diseases are presented. The MSC derived extracellular vesicles are provided in pharmaceutically acceptable preparations with a carrier, such as saline, and may be used to inhibit activation of antigen presenting cells. These preparations may also be used to suppress the development of T helper 1 (Th1) and Th17 cells. The disclosed activated MSC-derived extracellular vesicle preparations are essentially free of MSCs and other cells. Methods and preparations for treating and/or inhibiting the inflammatory response attendant organ transplant, diseases including human uveitis, type 1 diabetes, scleroderma, rheumatoid arthritis, lupus, Sjorgren's syndrome, spondyloarthritides, systemic sclerosis, systemic lupus erythematosus, antiphospholipid syndrome, multiple sclerosis, anti-glomerular basement membrane disease, and pemphigoid diseases, are also provided.

### CROSS-REFERENCE TO RELATED APPLICATIONS

This Application claims priority to U.S. Provisional Patent Application No. 62/549,892 filed on Aug. 24, 2017 to Ryang Hwa LEE, Joo Youn OH, Darwin J. PROCKOP, Dong-Ki KIM and Taek KURODA, currently pending, the entire disclosure of which is incorporated herein by reference.

### GOVERNMENT RIGHTS TO THE INVENTION

This invention was made with government support under grant number: P40RR17447 awarded by the National Institutes of Health. The government has certain rights in this invention.

### FIELD OF THE INVENTION

The invention relates to the field of extracellular vesicles produced by mesenchymal stem/stromal cells (MSC), and pharmaceutical preparations that comprise these extracellular vesicles. The invention also relates to the field of therapeutic methods, particularly methods for treating autoimmune diseases.

### BACKGROUND OF THE INVENTION

Mesenchymal stem/stromal cell (MSC)-based therapeutic intervention has become an emerging strategy for immune modulation, and therefore, MSCs have been exploited in a variety of clinical trials for immune-mediated disorders including autoimmune diseases. Although the exact mechanisms underlying the immunomodulatory functions of MSCs remain largely unknown, MSCs have shown suppressive effects on many types of immune cells in vitro and in vivo. For example, it has been reported that MSCs directly suppress T cell activation/proliferation and induced T cell apoptosis by expressing nitric oxide (NO), indoleamine 2,3, dioxygenase (IDO), programmed death ligand 1 (PD-L1) or Fas ligand (FASL) (Abdi et al., 2008; Akiyama et al., 2012; Jurewicz et al., 2010; Lee et al., 2011; Lenardo et al., 1999; Meisel et al., 2004; Sato et al., 2007; Wei et al., 2013). Also, MSCs have been shown to affect differentiation, maturation, and function of antigen presenting cells (APCs) including dendritic cells and macrophages, which results in conversion of APCs into a suppressive or tolerogenic phenotype (Aldinucci et al., 2010; Beyth et al., 2005; Chiesa et al, 2011; Jiang et al., 2005; Kronsteiner et al., 2011; Liu et al., 2013; Spaggiari et al., 2009; Zhang et al., 2009; Zhang et al., 2004).

Although MSC therapies are safe compared to embryonic stem cells or induced pluripotent stem cells which have tumorigenic potential, there are still concerns regarding allo-immune responses and pulmonary embolism that MSCs might trigger in a clinical setting (Ankrum et al., 2014; Barkholt et al., 2013; Boltze et al., 2015; Heslop et al., 2015; Isakova et al., 2014; Jung et al., 2013). In line with these clinical findings, intravenous administration of MSCs has been reported to cause embolism and death in mice (Furlani et al., 2009; Lee et al., 2009b; Tatsumi et al., 2013). Therefore, the long-term safety of MSC administration remains questionable.

Challenges continue to exist for use of MSC's or EVs in therapeutic applications. For example, EVs are highly heterogeneous depending on the cellular source, state and environmental condition. In addition, MSCs isolated from different donors have been reported to exhibit variation in their therapeutic efficacy in suppressing inflammation in vivo. Some MSCs failed to show any therapeutic effects altogether in sterile inflammation-mediated disease models (Lee et al., 2014). It has been observed that the therapeutic efficacy of MSCs in suppressing sterile inflammation correlates with the TSG-6 mRNA level in MSCs (Lee et al., 2014).

It has been reported that treatment using extracellular vehicles (EVs) have advantages over cell therapy. One reported advantage is that EVs are stable in the circulation without losing function and exhibit a superior safety profile over some forms of cell therapy (Vader et al., 2016). MSCs are an attractive source of EVs because they secrete a large number of therapeutic factors., including cytokines, chemokines, and microRNAs (Aggarwal and Pittenger, 2005; Baglio et al., 2015; Jurewicz et al., 2010; Lee et al., 2011; Meisel et al.,2004; Phinney et al., 2015; Rafei et al., 2008; Sato et al., 2007; Wei et al., 2013). In addition, MSCs have a tendency to infiltrate to injured tissues (Kidd et al., 2009; Ortiz et al., 2003; Rojas et al., 2005). Some of the EVs produced by MSCs have been reported to retain a homing capacity. EVs produced by MSCs have also been reported to exert their therapeutic effects in several disease models (Chen et al., 2015; Doeppner et al., 2015; Heldring et al., 2015; Monsel et al., 2016; Ophelders et al., 2016; Rani et al., 2015; Vader et al., 2016; Wen et al., 2016).

Th1 cytokine production is characteristic of many organ-specific autoimmune diseases (Alleva et al., 2001; Crane and Forrester, 2005; Jun et al., 1999; Weaver et al., 2001). IL-17A and/or IL-17F are responsible for development of inflammation in autoimmune disease disorders (Bettelli et al., 2007; Jain et al., 2008; Langrish et al., 2005; Nakae et al., 2002). MSCs have been reported to induce immune tolerance by activating the endogenous immune regulatory system of recipients, and in this manner, suppress autoimmune responses in models of type 1 diabetes (T1D) (Kota et al., 2013) and experimental autoimmune uveoretinitis (EAU) (Ko et al., 2016; Lee et al., 2015; Oh et al., 2014). However, it was not known if extracellular vesicles derived from MSC are also potentially effective in modulating immune responses. For a number of reasons, including medical safety, EVs could provide a preferred and improved alternative to preparations of cells, such as MSCs, as a therapeutic regimen. A medical need continues to exist for alternatives to cell therapy for autoimmune disease prevention.

### SUMMARY OF THE INVENTION

In a general and overall sense, the present invention provides therapeutic preparations having pharmacological activity comprising an enriched population of extracellular vesicles (EV) derived from particular populations of activated mesenchymal stem/stromal cells (MSC), and methods of using these EVs derived from MSCs in pharmaceutical preparations for therapeutic treatments, particularly in the treatment of certain autoimmune diseases and/or the inflammatory response attendant these diseases.

In one aspect, the pharmaceutical preparations of MSC-derived EVs are provided in a method for treating autoimmune diseases. In particular embodiments, the autoimmune diseases include those diseases that affect numerous sites in the body, including the pancreas and eye, as well as systemic immune response disorders, including organ transplant rejection. In addition, methods of using the pharmaceutical preparations enriched for MSC-derived EVs as part of a more general treatment for suppression of Th1 development and inhibition of activation of APCs and T cells, and the various diseases attendant these types of responses, are also presented.

The pharmaceutical preparations are also employed in a preparation and method for increasing immunosuppressive cytokine IL-10 expression in vivo, as well as in preparations and methods for suppressing Th17 cell development in vivo. The present invention thus provides for the use of preparations comprising the enriched population of specifically defined MSC-derived EVs in treating autoimmune diseases through the effect of these preparations on Th1 and Th17 cells.

IL-10 has been described as an immunosuppressive cytokine because of its association with multiple suppressive immune-cell populations, such as Tregs and regulatory DCs, as well as its inhibition on antigen presentation and immune-cell activation (Ouyang et al., 2011; Zhang et al., 2016). Given the demonstration here of the increased IL-10 and the hypoactive phenotype of DCs at the early time point of the MLR (day 2), a highly specialized method for using MSC-derived EVs to suppress Th1 and Th17 cell development without inducing Tregs is provided. For example, the MSC-derived EVs are provided, wherein the preparation induces IL-10 expressing regulatory DCs, and thereby, the regulatory DCs subsequently suppress Th1 and Th17 cell development without inducing Tregs.

The immunosuppressive effect of MSCs are mediated by a range of immunosuppressive mediators such as NO, IDO, prostaglandin E2 (PGE2), TNFα-simulated gene 6 (TSG-6), CCL-2, or PD-L1 (Aggarwal and Pittenger, 2005; Jurewicz et al., 2010; Lee et al., 2011; Meisel et al., 2004; Rafei et al., 2008; Sato et al., 2007; Wei et al., 2013). Since MSCs need to be activated to increase the expression of these therapeutic factors by inflammatory cytokines such as TNF-α or IFN-γ (Lee et al., 2009a; Wei et al., 2013), EVs isolated from unactivated MSCs are likely to express lower levels of therapeutic factors. To obtain EVs for the present studies, MSCs were incubated in a chemically defined protein-free medium, which activates MSCs to increase therapeutic proteins, including TSG-6, and also provides a stable environment for producing EVs. Therefore, the specialized preparations of MSC-derived EVs produced as described herein possess advantages over the EVs produced by unactivated MSCs. It is also contemplated that MSC cultured in serum-free media would be a useful clinical grade therapeutic product.

The MSC-derived EV-treatment provided for the preservation of islet function in vivo. In addition, a decrease in islets demonstrating insulitis was demonstrated. More than a single treatment, such as two or more treatments, of the preparations, such as in administration of additional doses of the MSC-derived EV treatments, may be provided according to some embodiments of the invention until a desired therapeutic response in the patient is evidenced, as part of the therapeutic methods described herein. The optimization of injection frequency and dose is well within the ordinary skill of one trained in the clinical and/or pharmaceutical arts, and may be identified without more than an ordinary amount of routine trial and error, in an effort to keep the long-lasting immunomodulation effects of the MSC-derived EV preparations described here.

In some embodiments, the MSCs employed to prepare the MSC derived EVs of the present formulations, preparations and treatments, are those MSCs that express high levels of TSG-6. This specialized population of MSCs are selected to prepare pharmaceutical preparations comprising the EVs of the present methods and compositions. Therapeutic efficacy of MSC-derived EVs may, in some cases, correlate with the MSC parent cells, and the TSG-6 level in these parent MSCs used to generate the MSC-derived EVs can be also used as a biomarker to select the cell source for EV production. Hence, pre-selecting the most effective MSC cellular source for EV production will help to avoid variation in therapeutic efficacy of the particular MSC-derived EVs and be essential for successful clinical translation. However, the EVs produced by the MSCs provided levels of TSG-6 that have been reported to be sub-therapeutic levels of TSG-6. Lastly, defining the therapeutic factors responsible for the immunomodulation effect in the MSC-derived EVs will also help to develop a biomarker to select the effective MSC cellular source for the MSC-derived EV preparation and can provide a strategy to maximize their therapeutic efficacy. For example, manipulating the MSC cellular source may be conducted so as to select a parent MSC population that overexpresses a defined and desired therapeutic factors, and then using this selected MSC population as the parent MSC source for the production of the MSC-derived EVs of the present preparations and methods.

In yet another aspect, methods and preparations for treating and/or inhibiting the inflammatory response attendant many diseases, including but not limited to organ transplant, as well as diseases including human uveitis, type 1 diabetes. scleroderma, rheumatoid arthritis, lupus, Sjorgren's disease, spondyloarthritides, systemic sclerosis, systemic lupus erythematosus, antiphospholipid syndrome, multiple sclerosis, anti-glomerular basement membrane disease, and pemphigoid diseases.

### BRIEF DESCRIPTION OF THE FIGURES

FIG. 1A. MSCs and MSCs-derived EVs prevent onset of T1D in mice. Experimental scheme. On day 0, MSCs (1×10⁶ cells), EVs (3 μg or 30 μg), or vehicle control was intravenously infused immediately after injection of splenocytes from diabetic NOD mice into NOD/scid mice. On day 4, MSCs, MSC-derived EVs, or vehicle control was infused again. Mice were monitored for hyperglycemia. FIG. 1B. and FIG. 1C. Diabetes incidence. PBS (n=10); MSC-derived EVs (n=10); HBSS (n=10); MSCs (n=10). P value by Kaplan-Meier estimator

FIG. 2A. MSC-derived EVs suppress insulitis in islets. The animals from the study described in FIG. 1B were sacrificed at day 58 (EV-treated group) and day 50 (MSC-treated group) for tissue harvest and blood collection, respectively. Representative hematoxylin-eosin staining of the pancreases. Arrowheads indicate islet-infiltrating immune cells. The control pancreas (Con) was obtained from age-matched NOD/scid mice. FIG. 2B. Islet number in pancreas per a slide (50 mm²; the bar represents the mean+SD. ** p<0.01, *** p<0.001 by one-way ANOVA with Dunnett's Multiple Comparison Test) and insulitis scores (**** p<0.0001 by two-way ANOVA). Five slides per each mouse (three or five mice per each group) were analyzed. FIG. 2C. Expression of insulin in the plasma. The bar represents the mean+SD. * p<0.05, ** p<0.01 by one-way ANOVA with Tukey's Multiple Comparison Test. FIG. 2D. Representative immunofluorescence staining for insulin (green) and CD4 (red). Nuclei were counterstained with DAPI (blue). Arrows indicate expression of insulin and arrowheads indicate CD4 signals. Scale bar=100 μm. DAPI, 4′,6-diamidino-2-phenylindole.

FIG. 3A. MSCs and MSC-derived EVs prevent development of EAU in mice. Experimental scheme. On day 0, EAU was induced by subcutaneous IRBP injection and intraperitoneal Pertussis toxin injection. Right after induction, either MSCs (1×10⁶ cells) or MSC-derived EVs (30 μg containing 15×10⁹ EVs) were injected into tail vein. As a control, the same volume of PBS was injected. On day 21, the eyeballs and draining cervical lymph nodes were collected for assays. FIG. 3B. Representative microphotographs of hematoxylin-eosin staining of the eyes, and histological disease scores of retinal pathology. FIG. 3C. Representative microphotographs of CD3 immunostaining of the eyes, and quantitative data of the number of CD3+ cells infiltrating the retina and. vitreous cavity. Dot represents a single animal, and data are presented in mean±SD. * p<0.05, ** p<0.01, **** p<0.0001 by one-way ANOVA.

FIG. 4A. MSC-derived EVs suppress Th1 development in EAU mice. Real-time PCR assays of the eyes of the animals from FIG. 3A. Data (mean+SD) were obtained from six mice per group. FIG. 4B. Representative flow cytometry plots and quantitative results for Th1 and Th17 cells in cervical lymph nodes (CLNs) collected from animals as in FIG. 3A. Dot indicates a single animal in FIG. 4B. The bar represents the mean±SD. * p<0.05, ** p<0.01, ***p<0.001 by one-way ANOVA.

FIG. 5A. MSC-derived EVs suppress Th1 development in the MLR. Splenic Th1 cytokine expressions at day 5 (IFN-γ) and day 2 (IL-12 p70 and TNF-α) in the MLR with or without MSCs or MSC-derived EVs (n=3 or 4). Ratio of MSCs to splenocytes=1:15, 1:30, and 1:60. FIG. 5B. Th17 cytokine expressions at day 2 (IL-6; n=1) and day 5 (IL-6 and IL-17A/F; n=3) in the MLR with or without MSC-derived EVs. FIG. 5C. Representative flow cytometry plots of CD4⁺CD25⁺Foxp3⁺ cells in the MLR assay with or without MSC-derived EV treatment. The cells were first gated on CD4 expression, and further analyzed for the expression of CD25 and Foxp3. FIG. 5D. Expression of IL-10 at day 5 in the MLR with or without MSC-derived EVs (n=4). All values are means±SD. * p<0.05, p<0.01, *** p<0.001 by one-way ANOVA.

FIG. 6A. MSC-derived EVs suppress activation of APCs and T cells in the MLR. Representative flow cytometry plots (FIG. 6A-FIG. 6B) and quantification (FIG. 6C) of CD80, CD86, CD40, and MHC-II positive cells in CD11c positive cells on day 2 of the MLR assay with or without MSC-derived EV treatment. The cells were first gated on CD11c expression, and further analyzed for the expression of CD80, CD86, CD40, and MHC-II (n=3). FIG. 6D. Expression of IL-10 at day 2 in the MLR with or without MSC-derived EVs (n=3 or 4). FIG. 6E. Quantification of flow cytometry analysis of CD40, and MHC-II positive cells in CD11c positive cells on day 2 of the MLR assay with CD11c positive responder cells (n=3). FIG. 6F. Expression of IL-2 and IFN-γ in CD4 positive cells at day 2 upon CD3/28 bead stimulation (n=4). All values are means±SD. * p<0.05, p<0.01, *** p<0.001 by one-way ANOVA.

FIG. 7A. Time course of retinal pathology and the percentages of Th1 and Th17 cells in lymph nodes. On day 0, EAU was induced, and on days 7, 14, and 21, the eyes and lymph nodes were evaluated. FIG. 7B. Retinal pathology scoring of the retmawratt line after EU immunization. FIG. 7C representative pictures of the retina with time after EAU immunization. FIG. 7D. Cytometrical analysis of cervical lymph nodes (CLN) and popliteal lymph nodes (PLN) with time after EAU immunization.

FIG. 8A. Treg analysis in cervical lymph nodes and blood of mice treated with MSCs or EVs. Representative flow cytometry plots, and FIG. 8B. Quantitative results for Foxp3⁺CD4⁺ Tregs in cervical lymph nodes (CLNs) and peripheral blood collected from EAU mice treated with PBS, MSCs, or EVs. For controls, normal mice without EAU induction were used.

### DETAILED DESCRIPTION OF THE PREFERRED EMBODIMENT

While preferred embodiments have been shown and described herein, it will be apparent to those skilled in the art that such embodiments are provided by way of example only. Numerous variations, changes, and substitutions will now occur to those skilled in the art without departing from the spirit of the disclosure. It should be understood that various alternatives to the embodiments described herein may be employed in practicing the subject matter described herein.

Certain Definitions:

As used in the specification and the appended claims, the singular forms “a”, “an” and “the” include plural references unless the context clearly dictates otherwise. Thus for example, reference to “the method” includes one or more methods, and/or steps of the type described herein and/or which will become apparent to those persons skilled in the art upon reading this disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

As used herein, the term “or” is an inclusive “or” operator and is equivalent to the term “and/or” unless the context clearly dictates otherwise.

The term “based on” is not exclusive and allows for being based on additional factors not described, unless the context clearly dictates otherwise.

The meaning of “in” includes “in” and “on.”

As used herein, “stem cell” refers to a multipotent cell with the potential to differentiate into a variety of other cell types (which perform one or more specific functions), and have the ability to self-renew.

As used herein, “adult stem cells” refer to stem cells that are not embryonic stem cells. By way of example, the adult stem cells include mesenchymal stem cells, also referred to as mesenchymal stromal cells or MSC's.

As used herein, the terms “administering”, “introducing”, “delivering”, “placement” and “transplanting” are used interchangeably and refer to the placement of the extracellular vesicles of the technology into a subject by a method or route that results in at least partial localization of the cells and/or extracellular vesicles at a desired site. The cells and/or extracellular vesicles can be administered by any appropriate route that results in delivery to a desired location in the subject where at least a portion of the cells and/or extracellular vesicles retain their therapeutic capabilities. By way of example, a method of administration includes intravenous administration (i.v.).

As used herein, the term “treating” includes reducing or alleviating at least one adverse effect or symptom of a disease or disorder through introducing in any way a therapeutic composition of the present technology into or onto the body of a subject.

As used herein, “therapeutically effective dose” refers to an amount of a therapeutic agent (e.g., sufficient to bring about a beneficial or desired clinical effect). A dose could be administered in one or multiple administrations (e.g., 2, 3, 4, etc.). However, the precise determination of what would be considered an effective dose may be based on factors individual to each patient, including, but not limited to, the patient's age, size, type or extent of disease, stage of the disease, route of administration, the type or extent of supplemental therapy used, ongoing disease process, and type of treatment desired (e.g., cells and/or extracellular vesicles as a pharmaceutically acceptable preparation) for aggressive vs. conventional treatment.

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the term “pharmaceutical composition” refers to the combination of an active agent the subcellular vesicles, with, as desired, a carrier, inert or active, making the composition especially suitable for diagnostic or therapeutic use in vitro, in vivo, or ex vivo. As used herein, the terms “pharmaceutically acceptable” or “pharmacologically acceptable” refer to compositions that do not substantially produce adverse reactions, e.g., toxic, allergic, or immunological reactions, when administered to a subject. For example, normal saline is a pharmaceutically acceptable carrier solution.

As used herein, the terms “host”, “patient”, or “subject” refer to organisms to be treated by the preparations and/or methods of the present technology or to be subject to various tests provided by the technology.

The term “subject” includes animals, preferably mammals, including humans. In some embodiments, the subject is a primate. In other preferred embodiments, the subject is a human.

The following abbreviations are used throughout the present document:

Abbreviations:

MSC Mesenchymal Stem Cells

EV Extracellular Vesicles

MLR Allogeneic mixed lymphocyte reaction

EAU uveoretinitis

Methods of Treatment:

The therapeutic uses of MSC-derived EVs in vivo for use in treating or inhibiting autoimmune diseases, including but not limited to autoimmune diseases involving the pancreas and eye, are presented. For example, the therapeutic uses of the MSC-derived EVs presented includes methods and preparations for treating and/or inhibiting the inflammatory response attendant organ transplant, as well as other autoimmune diseases including diabetes, human uveitis, type 1 diabetes. scleroderma, rheumatoid arthritis, lupus, and Sjorgren's disease.

Preparations comprising EVs derived from specially selected populations of MSCs are presented, and act to suppress Th1 development and inhibit activation of APCs and T cells, increase immunosuppressive cytokine IL-10 expression and suppressed TH17 cell development. Cytokine production attendant organ-specific autoimmune diseases, in particular, is reduced and/or inhibited, and in this manner, provides for the inhibition of the development of inflammation associated with disorders in autoimmune disease. In particular, the present pharmaceutical preparations may be used as part of a clinical regimen for treating autoimmune diseases.

Specifically defined and selected MSC derived EV populations are here demonstrated to provoke an increase in IL-10 and in the hypoactive phenotype of DCs at an early time point of the MLR (day 2). These defined MSC-derived EV populations may therefore be used to induce IL-10 expressing regulatory DCs. In this manner, the regulatory DCs act to suppress Th1 and Th17 cell development without inducing Tregs.

The immunosuppressive effect of MSCs are mediated by a range of immunosuppressive mediators such as NO, IDO, prostaglandin E2 (PGE2), TNFα-simulated gene 6 (TSG-6), CCL-2 or PD-L1 (Aggarwal and Pittenger, 2005; Jurewicz et al., 2010; Lee et al., 2011; Meisel et al., 2004; Rafei et al., 2008; Sato et al., 2007; Wei et al., 2013). MSCs need to be activated to increase the expression of these therapeutic factors by inflammatory cytokines, such as TNF-α or IFN-γ (Lee et al., 2009a; Wei et al., 2013). Therefore, other preparations of EVs isolated from unactivated MSC preparations and/or MSC populations are likely to express lower levels of therapeutic factors, and therefore not be satisfactory for providing the therapeutic preparations provided here.

The specially defined and activated MSC-derived EVs disclosed here provide a novel and improved non-cell (i.e., essentially cell free) preparation that may be used as a therapeutic preparation for autoimmune diseases prevention and treatment.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

In order that the disclosure described herein may be more fully understood, the following examples are set forth. It should be understood that these examples are for illustrative purposes only and are not to be construed as limiting this invention in any manner.

### EXAMPLE 1

### Materials and Methods

The present example presents the methods as well as a description of materials employed throughout the examples.

Extracellular Vesicles Derived from Specifically Defined Activated Mesenchymal Stem/Stromal Cells:

MSCs were incubated in a chemically defined protein-free medium, which activates MSCs to increase therapeutic proteins, including TSG-6, and also provides a stable environment for producing EVs. A protein-free medium for culturing MSC's generally is described in Kim et al., 2016, which is specifically incorporated herein by reference.

The EV-treated mice showed the preserved islet function, but they still showed a decreased β-cell mass in association with insulitis. Therefore, additional EV treatments might be required to prevent the onset of disease. Optimization of any frequency injection and dose to maintain any long-lasting immunomodulation effects of EVs will be developed. EVs are highly heterogeneous depending on the cellular source, state and environmental condition.

MSCs isolated from different donors may exhibit variation in their therapeutic efficacy in suppressing inflammation in vivo. Some populations of MSCs fail to show any therapeutic effects in sterile inflammation-mediated disease models (Lee et al., 2014). Therapeutic efficacy of MSCs in suppressing sterile inflammation correlates with the TSG-6 mRNA level in MSCs (Lee et al., 2014).

MSCs expressing the highest levels of TSG-6 were selected to prepare the EVs of the present studies and preparations for treatment. The TSG-6 level in a parent MSC population may also be used as a biomarker to select a suitable MSC cell sources for therapeutic EV production according to the present invention. Pre-selecting the most effective MSC cellular source for EV production will reduce variation in therapeutic efficacy of the population of MSC-derived EVs for clinical translation. Defining the therapeutic factors responsible for the immunomodulation effect in the present selected EV preparations will also help to develop a biomarker to select the most effective MSC cellular source for EV preparation and can provide a strategy to maximize the therapeutic efficacy of the EV preparation produced. Manipulating the EV cellular source by overexpressing the defined therapeutic factors is one technique that can be used to provide MSC-derived EVs having enhanced therapeutic efficacy.

MSC Culture and Isolation of MSC-Derived EVs.

Human MSCs (donor #6015) were prepared as previously described (Lee et al., 2009a) and EVs derived from MSCs were prepared as previously described (Kim et al., 2016). In brief, a frozen vial of passage 3 to 4 MSCs was plated directly at about 200 to 500 cells per cm² in tissue culture plates in complete culture medium (CCM). The CCM medium was replaced after 2-3 days. After the cells reached about 70% confluency in 4-6 days, the MSCs were either harvested for mouse injections or incubated with a medium optimized for Chinese hamster ovary cells (CD-CHO Medium; Invitrogen; Thermo Fisher Scientific, Waltham, Mass.) with additional supplements (Kim et al., 2016) for EV production. After 6 h, the medium was discarded and the fresh medium was replaced and recovered at 48 h to isolate EVs.

For isolation of EVs, the medium was centrifuged at 2,565^{×}g for 15 min to remove cellular debris, and the supernatant was applied directly at room temperature to a column containing the anion exchange resin (Express Q; cat no. 4079302; Whatman;100-mL bed volume) that had been equilibrated with 50 mM NaCl in 50 mM Tris buffer (pH 8.0). The medium was applied at a flow rate of 4 ml/min and at room temperature. The column resin was washed with 10 volumes of the equilibration buffer and then eluted with 25 volumes of 500 mM NaCl in 50 mM Tris buffer (pH 8.0). Fractions of 20-30 mL were collected and stored at either −80° C. before in vitro and in vivo assays. The EVs in the peak fractions were positive for the exosome markers, CD63 and CD81, but negative for 11 other epitopes found on the MSCs from which they were recovered. Also, they were about 100 nm in diameter.

Adoptive Transfer Type 1 Diabetes (T1D) Mouse Model:

Female NOD/LtJ (12 weeks old) and female NOD/scid mice (7 weeks old) were used for adoptive transfer model. All mice were purchased from Jackson Laboratory (Bar Harbor, Me.) and cared for at Scott & White Department of Comparative Medicine under a protocol approved by the Institutional Animal Care and Use Committee. To induce an adoptive transfer in the T1D model, 10⁷ splenocytes from pre-diabetic 12-Week-old female NOD mice were intravenously injected into 7-week-old female NOD/scid mice. 1×10⁶ MSCs (#6015, the same lot of MSCs from which EVs were produced), EVs (15×10⁹ or 30 μg), or vehicle control were intravenously injected twice at 15 minutes and day 4 after splenocyte transfer. Blood glucose levels were measured twice a week by tail bleeding according to National Institutes of Health guidelines, and diabetes in mice was defined as having the two consecutive glycemic values above 250 mg/dL.

Pancreas Histology After Adaptive Transfer in Type 1 Diabetes (T1D) Model:

Serial pancreatic sections (5 μm) were prepared from at least three mice per each group. Every 20^{th} sections (n=5) were stained with hematoxylin-eosin (H-E) and islet number per a section (about 50 mm² area) was quantified. Insulitis scoring was performed on H-E-stained pancreatic sections as we have shown previously (Kota et al., 2013). Briefly, insulitis was scored as follows: grade 0, normal islets; grade 1, mild mononuclear infiltration (<25%) at the periphery; grade 2, 25-50% of the islets infiltrated; grade 3, >50% of the islets infiltrated; grade 4, islets completely infiltrated with no residual parenchyma remaining. For immunofluorescence, the sections were incubated for 18 h at 4° C. with antibodies against mouse insulin (1:800, clone C27C9; Cell Signaling, Danvers, Mass.) and mouse CD4 (1:100, YTS191.1; Bio-Rad Laboratories, Hercules, Calif.).

Human Uveoretinitis—EAU Mouse Model:

The protocols employed were approved by the Institutional Animal Care and Use Committee of Seoul National University Biomedical Research Institute (IACUC No. 13-0104-C1A1). Six-week-old female B6 mice (C57BL/6J, H-2b; Orient Bio, Seongnam, Korea) were immunized with subcutaneous injection into a footpad of the retina-specific antigen, interphotoreceptor retinal binding protein (IRBP) peptide 1-20, GPTHLFQPSLVLDMAKVLLD (250 μg; Peptron, Daejeon, Korea) emulsified in complete Freund adjuvant (Sigma-Aldrich, Saint Louis, Mo.) containing Mycobacterium tuberculosis (2.5 mg/ml; BD Difco, Franklin Lakes, N.J.). Simultaneously, the mice received intraperitoneal injection of 0.7 μg pertussis toxin (300 μl; Sigma-Aldrich). Immediately after immunization, MSC-derived EVs (15×10⁹ or 30 μg of EVs) in 150 μl of PBS, 1×10⁶ MSCs (#6015, the same lot of MSCs from which EVs were produced) in 150 μl PBS, or the same volume of PBS were injected via tail vein into the mice.

Eyeball Histology

Twenty one days later, the mice were humanely killed, and eyeballs were collected for assays. Eyeballs were subjected to histological and molecular assays. For histology, the eyeballs were fixed in 10% formaldehyde and embedded in paraffin. Serial 4 μm thick sections were cut and stained with hematoxylin-eosin and CD3 immunohistochemical staining. For CD3 immunohistochemical staining, a rabbit anti-mouse CD3 (ab5690, Abcam, Cambridge, Mass.) was used as a primary antibody. The pathologic features of the retina were examined, and histological disease score was assessed by two independent observers (JYO and TWK) in a blinded manner on a scale of 0 to 4 using the criteria previously defined by Caspi (Caspi, 2003). The number of CD3-stained cells was calculated under a microscope using ×20 object.

Allogeneic Mixed Lymphocyte Reaction (MLR)

MSCs or EVs were co-cultured in 96-well plates with splenocytes from BALB/c mice (0.3 M cells/well) and C57BL/6 mice (0.6 M cells/well) in 5% heat-inactivated FBS (Atlanta Biologicals, Flowery Branch, Ga.) plus 100 units/ml penicillin and 100 mg/ml streptomycin (pen/strep; both from Life Technologies, Carlsbad, Calif.) in RPMI-1640 medium (ATCC, Manassas, Va.). All mice were purchased from Jackson Laboratory. MSCs and splenocytes from BALB/c mice were pretreated with mitomycin (2.5 mg/ml for 2 h at 37° C.; Sigma-Aldrich) before co-culture. Two days or five days later, mouse cytokine expressions were measured by real-time PCR assays or ELISAs according to the manufacture's protocols.

Isolation and Activation of T Cells

CD4⁺ T cells were isolated from splenocytes from BALB/c mice by CD4⁺ T Cell Isolation Kit II (Miltenyi Biotec, San Diego, Calif.) according to the manufacture's protocol. The CD4⁺ T cells were cultured in 96-well plates with CD3/CD28 beads (Life Technologies) with or without EVs in RPMI-1640 medium containing 5% heat-inactivated FBS, 100 units/ml penicillin and 100 mg/ml streptomycin. Two days later, the levels of T helper 1 (Th1) cytokines were detected by ELISA according to the manufacture's protocols.

Flow Cytometry Analysis

Cervical draining lymph nodes (CLNs) from mice were analyzed for Th1, Th17, and regulatory T cells (Tregs) by flow cytometry at 21 days after EAU induction. For flow cytometry, CLNs were minced between the frosted ends of two glass slides to obtain a single-cell suspension in RPMI-1640 medium (WelGENE, Daegu, Korea) containing 10% FBS (Gibco; Life Technologies). The cells were stained with fluorescence-conjugated anti-mouse antibodies against CD4, Foxp3, IFN-γ (all from eBioscience, San Diego, Calif.) and IL-17A (BD Pharmingen™, San Diego, Calif.). IN-γ (XMG1.2; BO Pharmingen, San Diego, Calif.). For intracellular staining, the cells were stimulated for 5 h with 50 ng/ml phorbol myristate acetate and 1 μg/mlionomycin in the presence of GolgiPlug (BO Pharmingen) and stained. The cells were then assayed for fluorescence using S1000EXi Flow Cytometer (Stratedigm, San Jose, Calif.). Data were analyzed using Flowjo program (Tree Star, Ashland, Oreg.).

EV-treated APC phenotypes in the MLR were analyzed by flow cytometry using anti-mouse CD11b (M1/70), CD11c (HL3), CD80 (16-10A1), CD86 (GL1), CD40 (3/23), and major histocompatibility complex (MHC) class II (1-A/1-E; M5/114.15.2) antibodies and all antibodies are from BD Biosciences (San Jose, Calif.). Mouse Treg Detection Kit (Miltenyi Biotec) was used to stain regulatory T cells (Tregs) for flow cytometry analysis.

Real-Time PCR Assay

For molecular assays, the eyeballs were lysed in RNA isolation reagent (RNA Bee; Tel-Test, Friendswood, Tex.) and homogenized using a sonicator (Ultrasonic Processor; Cole Parmer Instruments, Vernon Hills, Ill.). Total RNA was extracted from the eyeballs or splenocyte culture using RNeasy Mini kit (Qiagen, Valencia, Calif.), and double-stranded cDNA were synthesized by reverse transcription (High Capacity RNA-to-cDNA Kit; Applied Biosystems; Life Technologies). Real-time PCR amplification (ABI 7900 Sequence Detector; Applied Biosystems) was performed using TaqMan Universal PCR Master Mix (Applied Biosystems). PCR probe and primer sets were purchased from Applied Biosystems (TaqMan Gene Expression Assay): IL-1β, IL-4, IL-10, IL-6, IL-12A, IL-17A, and IFN-γ. For relative quantitation of gene expression, mouse-specific GAPDH primers and probe (Mm99999915_g1) were used.

ELISA

Mouse insulin in the plasma from NOD/scid mice of T1D model was detected by Mouse INSULIN ELISA Kit (EMINS; Thermo Fisher Scientific). Mouse IFN-γ, IL-2, IL-10 and IL-12 in the culture supernatants were measured by commercial ELISA Kits (IFN-γ: DY485; IL-2: DY402; IL-10: M1000B; L-12 p70: M1270; R&D Systems, Minneapolis, Minn.) according to the manufacture's protocol.

### EXAMPLE 2

### MSC-Derived EVs Delay Onset of Type 1 Diabetes (T1D) In Vivo

The present example demonstrates the immunosuppressive capacity of the specifically defined MSC-derived EVs in vivo. In addition, the immunosuppressive effect of the present preparations in animals with T1D is shown.

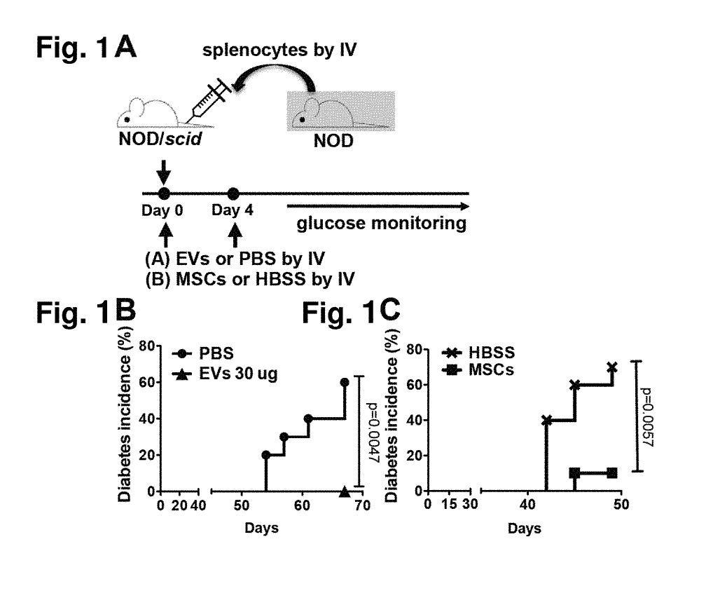

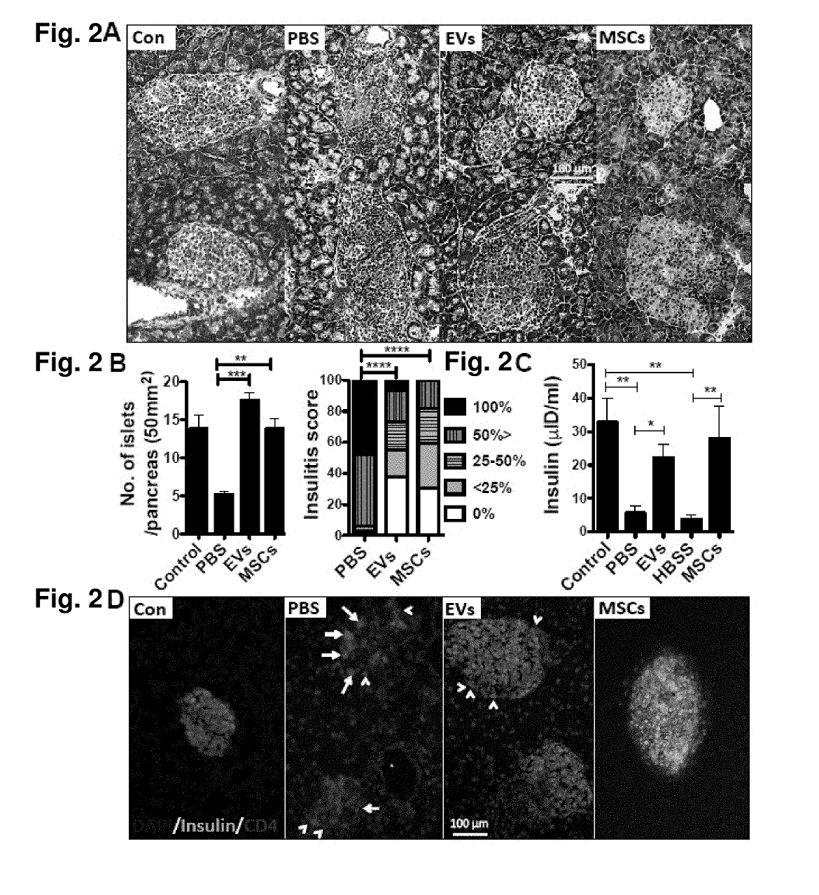

To induce an adoptive transfer T1D model, splenocytes isolated from 12-week-old female NOD mice were intravenously infused into 7-week-old female NOD/scid mice (FIG. 1A). To test the effects of MSC-derived EVs, either 1) MSC-derived EVs (30 μg containing 15×10⁹ EVs per mouse or a vehicle control (PBS) was injected, or 2) MSCs (1×10⁶ cells per mouse, donor #6015, the same lot of MSCs from which EVs were produced) or their vehicle control (HBSS was injected into tail vein right after adoptive splenocyte transfer. Mice received an additional treatment at day 4 as shown in FIG. 1A. Recipient NOD/scid mice were monitored for hyperglycemia twice a week, and diabetes development was defined as the mouse having the glycemic value of above 250 mg/dL. As shown in FIG. 1B, both of MSC-derived EVs and MSCs significantly delayed the onset of T1D in an adoptive transfer T1D model. Histologic analysis revealed that most of the islets were already destroyed at day 58, and the remaining islets showed severe insulitis in the PBS-treated mice (FIGS. 2A, 2B, and 2D). In contrast, administrations of MSC-derived EVs or MSCs suppressed insulitis and preserved insulin-producing cells in the islets (FIGS. 2A, 2B, and 2D). In addition, there were fewer CD4⁺ cells in islets of EV- or MSC-treated mice while CD4⁺ cells were present in significant numbers in the PBS-treated mouse islets (FIG. 2D). Consistent with these histologic results, the plasma levels of insulin were significantly increased by treatment with either EVs or MSCs (FIG. 2C). These results demonstrated that MSC-derived EVs were as effective in delaying the onset of T1D in mice as MSCs.

### EXAMPLE 3

### MSC-Derived EVs Prevent Development of Uveitis

The present example demonstrates the utility of the invention for providing a treatment for human endogenous uveitis.

Experimental autoimmune uveitis (EAU) is an animal disease model of human endogenous uveitis was used. This model can be induced in susceptible animals by immunization with retinal antigens (Ags). Ocular antigens (Ags) such as uveal melanin and proteins involved in its metabolism, like retinal arrestin (retinal soluble antigen or [S—Ag]), inter-photoreceptor retinoid-binding protein (IRBP), and recoverin, are used to immunize animals so as to induce uveitis.

Several animal models of uveitis have been described. Endotoxin induced uveitis is another useful model for anterior uveitis, which is not an autoimmune process and is triggered by injection of bacterial endotoxin (lipopolysaccharides) resulting in a rapid short lasting uveitis.

Uveitis is a general term used for the inflammation of the uveal tissue (iris, ciliary body, and choroid). Anatomically it has been classified as anterior, intermediate and posterior or as panuveitis. Noninfectious uveitis is believed to be autoimmune or immune-mediated. Although the distinction between autoimmune and immune-mediated uveitis is still indistinct, the autoimmune type is believed to be driven by aberrant immune recognition of self, whereas the immune-mediated is primarily an inflammatory reaction triggered by environmental (microbial) or autologous (tissue damage) signals. Uveitis, especially if untreated, can result in significant visual deficit and blindness. It accounts for 5-20% of blindness in the developed countries and 25% in the developing countries.

In idiopathic uveitis, the possible mechanism hypothesized is of molecular mimicry with common micro-organisms, but the etiological triggers in autoimmune uveitis are unknown. However, strong major histocompatibility complex (MHC) associations have been found to be linked with some of the different types of autoimmune uveitis

In parallel studies, the effects of MSC-derived EVs was examined in a mouse model of EAU (Ko et al., 2016), a well-established model for human autoimmune intraocular inflammation, and compared with the effects of MSCs. Briefly, mice were immunized with s.c. injection into a footpad of 250 μg human IRBP peptide 1-20, GPTHLFQPSLVLDMAKVLLD (20 mg/mL; Peptron), that was emulsified in complete Freund adjuvant (Sigma-Aldrich) containing Mycobacterium tuberculosis (2.5 mg/mL; BD Difco). Simultaneously, the mice received i.p. injection of 0.7 μg pertussis toxin (300 μL; Sigma-Aldrich).

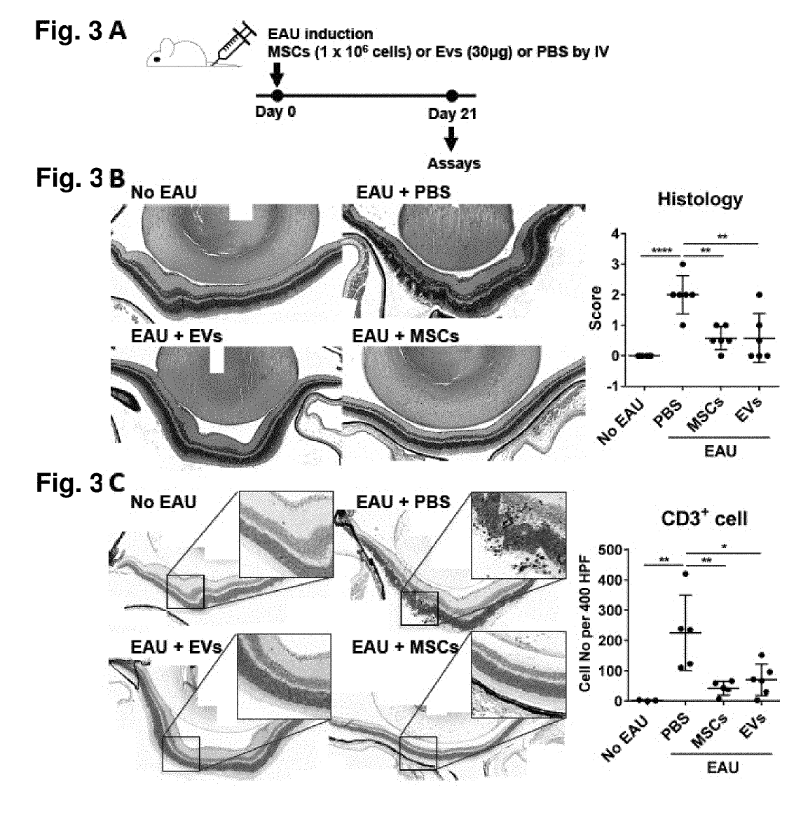

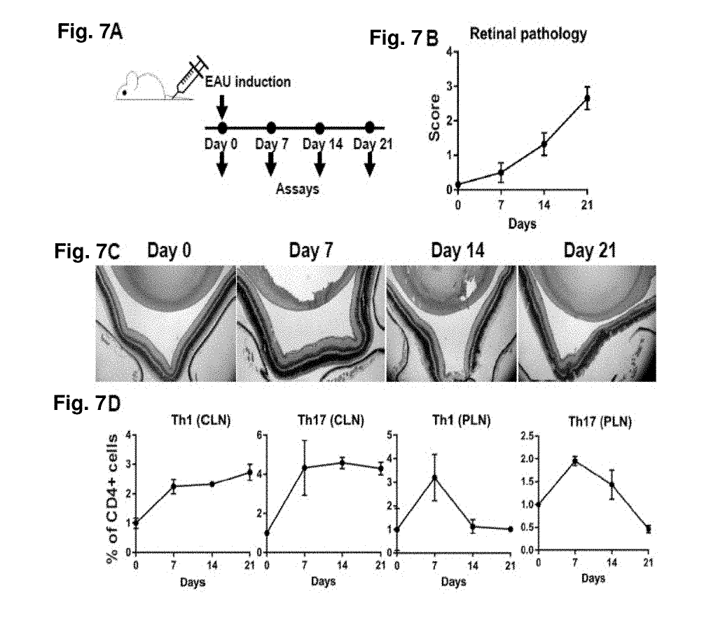

Immediately after EAU immunization (day 0), one of the following treatments were administered: 1) MSC-derived EVs (30 μg containing 15×10⁹ EVs per mouse), 2) MSCs (1×10⁶ cells per mouse, donor #6015, the same lot of MSCs from which EVs were produced), or 3) their vehicle control (PBS) through tail vein injection (FIG. 3A). The mice were sacrificed at day 21, and the eyes and CLNs were assayed. The day 21 time-point was selected for evaluation because in previous time course experiments, it was found that both the retinal destruction and Th1/Th17 activation in CLNs were at peak (FIG. 7). The retinal cross-sections at day 21 showed severe disruption of retinal photoreceptor layer and infiltration of inflammatory cells including CD3⁺ T cells in the retina and vitreous cavity in EAU mice treated with PBS (FIG. 3B and FIG. 3C). In contrast, there was little structural damage with few inflammatory infiltrates and in the eyes of EAU mice received MSCs or MSC-derived EVs, similar to the normal retina without EAU induction (FIG. 3B). The disease score assigned by retinal pathology was significantly lower in MSC- or MSC-derived EV-treated mice compared to the PBS-treated mice (FIG. 3B). Also, the number of CD3⁺ T cells infiltrating the retina was significantly reduced by either MSCs or MSC-derived EVs (FIG. 3C). There were no differences in the disease score and the number of infiltrating CD3⁺ cells between MSC-derived EV- and MSC-treated groups.

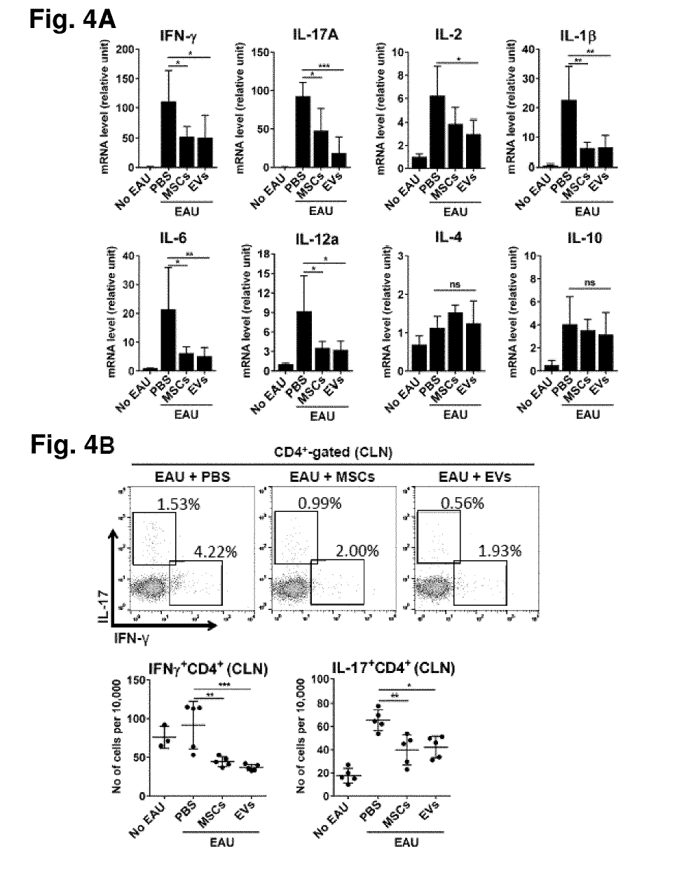

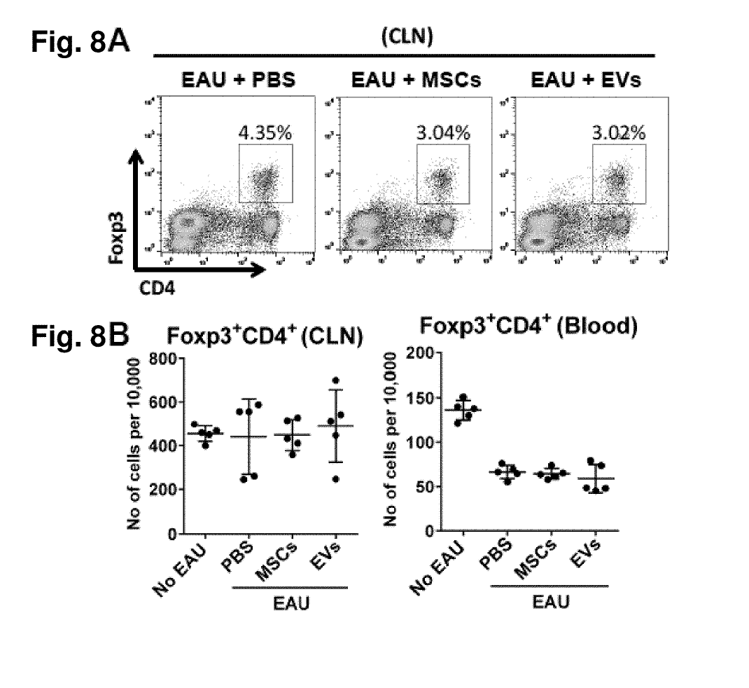

The transcript levels of pro-inflammatory cytokines, IFN-γ, IL-17A, IL-2, IL-1β, IL-6, and IL-12A were significantly lower in the eyes of MSC- or MSC-derived EV-treated group animals compared with the PBS-treated control animals (FIG. 4A). However, the mRNA levels of IL-4 and IL-10 were not affected by treatment (FIG. 4A). The effects of MSC-derived EVs in the reduction of inflammatory markers were comparable to those of MSCs. In addition, flow cytometric assays of CLNs revealed the number of IFN-γ⁺CD4⁺ cells and IL-17⁺CD4⁺ cells was significantly lower in MSC or MSC-derived EV-treated mice than in the PBS-treated mice (FIG. 4B). The number of Foxp3⁺ Tregs was not different between all groups (FIG. 8). Together, these data indicate that MSC-derived EVs are as effective in suppressing Th1 and Th17 cells and preventing EAU development as their parent MSC cells.

### EXAMPLE 4

### Activated MSC-Derived EVs Suppress T Cell Proliferation in Allogeneic Mixed Lymphocyte Reaction (MLR)

The present example is provided to demonstrate the utility of the present preparations for suppressing T-cell proliferation.

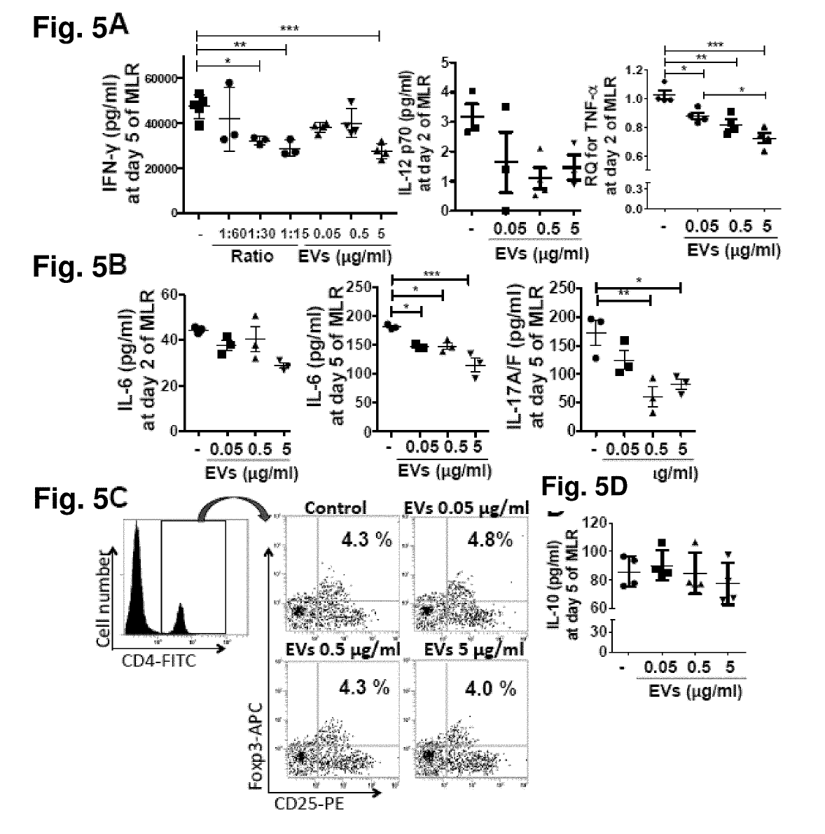

To demonstrate the activity of the present preparations in reference to the underlying mechanism of their role in modulating immune response, the effects of the specially defined activated MSC-derived EVs on immune cell activation using allogeneic MLR assays is demonstrated. The specially defined activated MSC-derived EVs significantly reduced the production of IFN-γ, IL-12 p70, and TNF-α in the MLR (FIG. 5A). This demonstrates that the specially activated MSC-derived EVs suppress Th1 development. In addition, the specially activated MSC-derived EVs significantly suppressed production of IL-6. IL-6 is a key cytokine for the lineage commitment of pathogenic IL-17 producing Th17 cells, as well as IL-17 in the MLR. The present data indicates that the specially derived MSC-derived EVs also suppress Th17 development (FIG. 5B).

Whether the specially activated MSC-derived EVs suppress Th1 and Th17 developments by inducing Tregs was also examined. There was no increase in Foxp3⁺ Tregs on day 6 of the MLR (FIG. 5C) and IL-10, a cytokine that induces Tregs, on day 5 of the MLR (FIG. 5D), indicating that the specially derived MSC produced EVs suppressed T cell proliferation by directly inhibiting Th1 and Th17 development, not by inducing Tregs.

### EXAMPLE 5

### MSC-Derived EVs Suppress Activation of APCs and T Cells

The present example demonstrates the utility of the present invention for suppressing the activation of APCs and T cells.

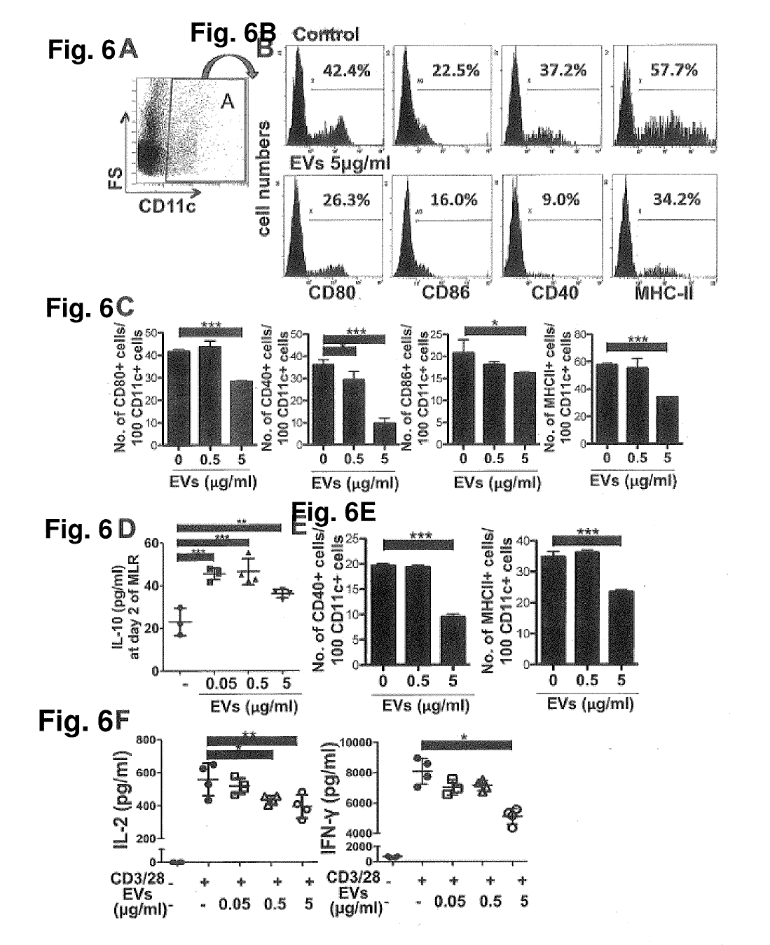

To investigate the effects of EVs on APC activation, the expression of costimulatory factors (CO80, CD86, and CD40) and MHC class II (MHC-II) in APCs cultured in the presence of the specially derived MSC-produced EVs was examined. The results showed that the present MSC-derived EV preparations provided a treatment that suppressed the expression of costimulatory factors and MHC-II in CD11c⁺ cells on day 2 of the MLR in a dose-dependent manner (FIGS. 6A, 68, and 6C). Also, the MSC-derived EV treatment significantly increased the levels of IL-10 on day 2 of the MLR (FIG. 6D).

To examine whether the MSC-derived EV preparations created here directly suppress APC activation, the MLR was repeated with whole splenocytes isolated from BALB/c mice as stimulator cells and only CD11c⁺ cells isolated from C57BL/6 mouse splenocytes as responder cells. As shown in FIG. 6E, treatment with the MSC-derived EV preparations still suppressed the expression of costimulatory factors and MHC-II in CD11c⁺ cells. These data suggest that APCs exhibit a hypoactive phenotype including the suppressed allorecognition and thereby, suppress subsequent T cell proliferation in the MLR.

To further examine whether the specially treated MSC derived EVs also directly inhibit T cell activation, CD4⁺ T cells were isolated from mouse splenocytes and were stimulated with CD3/CD28 beads. The results showed that treatment with the specific preparation of MSC derived EVs also suppressed T cell activation as indicated by decreased levels of IL-2 and IFN-γ (FIG. 6F). Together, these data demonstrate that the specially described and derived MSC-derived EV preparations suppress activation of both APCs and T cells in the MLR.

It is intended that the following claims define the scope of the disclosure and that methods and structures within the scope of these claims and their equivalents be covered thereby.

### BIBLIOGRAPHY

Abdi, R., Fiorina, et al., (2008). Diabetes 57, 1759-1767.
Aggarwal, S., and Pittenger, M. F. (2005). Blood 105, 1815-1822.
Akiyama, K., et al., (2012). Cell. Stem Cell. 10, 544-555.
Aldinucci, A., et al., (2010). J. Immunol. 185, 5102-5110.
Alleva, D. G., et al., (2001). J. Leukoc. Biol. 69, 440-448.
Ankrum, J. A., Ong, J. F., and Karp, J. M. (2014). Nat. Biotechnol. 32, 252-260.
Baglio, S. R., et al. (2015). Stem Cell. Res. Ther. 6, 127-015-0116-z.
Barkholt, L., et al. (2013). Cytotherapy 15, 753-759.
Bettelli, E., Oukka, M., and Kuchroo, V. K. (2007). Nat. Immunol. 8, 345-350.
Beyth, S., et al. (2005). Blood 105, 2214-2219.
Boltze, J., et al. (2015). Front. Neurol. 6, 155.
Caspi, R. R. (2003). Experimental autoimmune uveoretinitis in the rat and mouse. Curr. Protoc. Immunol. Chapter 15, Unit 15.6.
Chen, J., Li, C., and Chen, L. (2015). Biomed. Res. Int. 2015, 985814.
Chiesa, S., et al. (2011). Proc. Natl. Acad. Sci. U.S.A. 108, 17384-17389.
Crane, I. J., and Forrester, J. V. (2005). Crit. Rev. Immunol. 25, 75-102.
Doeppner, T. R., et al. (2015). Stem Cells Transl. Med. 4, 1131-1143.
Furlani, D., et al. (2009). Microvasc. Res. 77, 370-376.
Heldring, N., et al. (2015). Hum. Gene Ther. 26, 506-517.
Heslop, J. A., et al., (2015). Stem Cells Transl. Med. 4, 389-400.
Isakova, I. A., et al. Allo-reactivity of mesenchymal stem cells in rhesus macaques is dose and haplotype dependent and limits durable cell engraftment in vivo. PLoS One 9, e87238.
Jain, R., Tartar, et al. (2008). J. Exp. Med. 205, 207-218.
Jiang, X. X., Zhang, Y., Liu, B., Zhang, S. X., Wu, Y., Yu, X. D., and Mao, N. (2005). Blood 105, 4120-4126.
Jun, H. S., Yoon, C. S., Zbytnuik, L., van Rooijen, N., and Yoon, J. W. (1999). J. Exp. Med. 189, 347-358.
Jung, J. W., Kwon, M., Choi, J. C., Shin, J. W., Park, I. W., Choi, B. W., and Kim, J. Y. (2013). Yonsei Med. J. 54, 1293-1296.
Jurewicz, M., Yang, S., Augello, A., Godwin, J. G., Moore, R. F., Azzi, J., Fiorina, P., Atkinson, M., Sayegh, M. H., and Abdi, R. (2010). Diabetes 59, 3139-3147.
Kidd, S., Spaeth, E., Dembinski, J. L., Dietrich, M., Watson, K., Klopp, A., Battula, V. L., Weil, M., Andreeff, M., and Marini, F. C. (2009). Stem Cells 27, 2614-2623.
Kim, D. K., Nishida, H., An, S. Y., Shetty, A. K., Bartosh, T. J., and Prockop, D. J. (2016). Proc. Natl. Acad. Sci. U.S.A. 113, 170-175.
Kimura, A., Naka, T., and Kishimoto, T. (2007). Proc. Natl. Acad. Sci. U.S.A. 104, 12099-12104.
Ko, J. H., Lee, H. J., Jeong, H. J., Kim, M. K., Wee, W. R., Yoon, S. O., Choi, H., Prockop, D. J., and Oh, J. Y. (2016). Proc. Natl. Acad. Sci. U.S.A. 113, 158-163.
Kota, D. J., Wiggins, L. L., Yoon, N., and Lee, R. H. (2013). Diabetes 62, 2048-2058.
Kronsteiner, B., et al. (2011). Cell. Immunol. 267, 30-38.
Langrish, C. L., et al. (2005). J. Exp. Med. 201, 233-240.
Lee, H. J., et al. (2015). J. Immunol. 194, 3634-3645.
Lee, R. H., Oh, J. Y., Choi, H., and Bazhanov, N. (2011). J. Cell. Biochem. 112, 3073-3078.
Lee, R. H., et al. (2009a). Cell. Stem Cell. 5, 54-63.
Lee, R. H., Seo, M. J., Pulin, A. A., Gregory, C. A., Ylostalo, J., and Prockop, D. J. (2009b). Blood 113, 816-826.
Lee, R. H., et al. (2014). Proc. Natl. Acad. Sci. U.S.A. 111, 16766-16771.
Lenardo, M., et al. (1999). Annu. Rev. Immunol. 17, 221-253.
Liu, W. H., Liu, J. J., Wu, J., Zhang, L. L., Liu, F., Yin, L., Zhang, M. M., and Yu, B. (2013). PLoS One 8, e55487.
Meisel, R., Zibert, A., Laryea, M., Gobel, U., Daubener, W., and Dilloo, D. (2004). Blood 103, 4619-4621.
Monsel, A., Zhu, Y. G., Gudapati, V., Lim, H., and Lee, J. W. (2016). Expert Opin. Biol. Ther. 1-13.
Nakae, S., Komiyama, Y., Nambu, A., Sudo, K., Iwase, M., Homma, I., Sekikawa, K., Asano, M., and Iwakura, Y. (2002). Immunity 17, 375-387.
Oh, J. Y., Kim, T. W., Jeong, H. J., Lee, H. J., Ryu, J. S., Wee, W. R., Heo, J. W., and Kim, M. K. (2014). Mediators Inflamm. 2014, 624640.
Ophelders, D. R., Wolfs, T. G., Jellema, R. K., Zwanenburg, A., Andriessen, P., Delhaas, T., Ludwig, A. K., Radtke, S., Peters, V., Janssen, L., Giebel, B., and Kramer, B. W. (2016). Stem Cells Transl. Med. 5, 754-763.
Ortiz, L. A., Gambelli, F., McBride, C., Gaupp, D., Baddoo, M., Kaminski, N., and Phinney, D. G. (2003). Proc. Natl. Acad. Sci. U.S.A. 100, 8407-8411.
Ouyang, W., Rutz, S., Crellin, N. K., Valdez, P. A., and Hymowitz, S. G. (2011). Annu. Rev. Immunol. 29, 71-109.
Phinney, D. G., et al. (2015). Nat. Commun. 6, 8472.
Rafei, M., et al. (2008). Blood 112, 4991-4998.
Rani, S., Ryan, A. E., Griffin, M. D., and Ritter, T. (2015). Mesenchymal Stem Cell-derived Extracellular Vesicles: Toward Cell-free Therapeutic Applications. Mol. Ther. 23, 812-823.
Rojas, M., Xu, J., Woods, C. R., Mora, A. L., Spears, W., Roman, J., and Brigham, K. L. (2005). Am. J. Respir. Cell Mol. Biol. 33, 145-152.
Sato, K., Ozaki, K., Oh, I., Meguro, A., Hatanaka, K., Nagai, T., Muroi, K., and Ozawa, K. (2007). Blood 109, 228-234.
Spaggiari, G. M., Abdelrazik, H., Becchetti, F., and Moretta, L. (2009). Blood 113, 6576-6583.
Tatsumi, K., Ohashi, K., Matsubara, Y., Kohori, A., Ohno, T., Kakidachi, H., Horii, A., Kanegae, K., Utoh, R., Iwata, T., and Okano, T. (2013). Biochem. Biophys. Res. Commun. 431, 203-209.
Vader, P., Mol, E. A., Pasterkamp, G., and Schiffelers, R. M. (2016). Adv. Drug Deliv. Rev.
Weaver, D. J., Jr, Poligone, B., Bui, T., Abdel-Motal, U. M., Baldwin, A. S., Jr, and Tisch, R. (2001). J. Immunol. 167, 1461-1468.
Wei, X., Yang, X., Han, Z. P., Qu, F. F., Shao, L., and Shi, Y. F. (2013). Acta Pharmacol. Sin. 34, 747-754.
Wen, S., Dooner, M., Cheng, Y., Papa, E., Del Tatto, M., Pereira, M., Deng, Y., Goldberg, L., Aliotta, J., Chatterjee, D., et al. (2016). Leukemia
Zhang, B., Liu, R., Shi, D., Liu, X., Chen, Y., Dou, X., Zhu, X., Lu, C., Liang, W., Liao, L., Zenke, M., and Zhao, R. C. (2009). Blood 113, 46-57.
Zhang, H., Wang, Y., Hwang, E. S., and He, Y. W. (2016). Interleukin-10: J. Clin. Oncol.
Zhang, W., Ge, W., Li, C., You, S., Liao, L., Han, Q., Deng, W., and Zhao, R. C. (2004). Stem Cells Dev. 13, 263-271.

We claim:

1. A method for inhibiting onset of an autoimmune disease in an animal comprising:
providing a therapeutically effective amount of a pharmaceutically acceptable preparation of extracellular vesicles derived from an activated preparation of mesenchymal stem cells to an animal; and
inhibiting onset of the autoimmune disease in the animal.

2. The method of claim 1 wherein the activated mesenchymal stem cells express a high level of TSG-6.

3. The method of claim 1 wherein the autoimmune disease is human uveitis, type 1 diabetes, scleroderma, rheumatoid arthritis, lupus, Sjorgren's syndrome, spondyloarthritides, systemic sclerosis, systemic lupus erythematosus, antiphospholipid syndrome, multiple sclerosis, anti-glomerular basement membrane disease, pemphigoid diseases, and autoimmune response to an organ transplant.

4. The method of claim 1 wherein the autoimmune disease is an autoimmune response to an organ transplant in the animal.

5. A pharmacologically active preparation of extracellular vesicles, said extracellular vesicles having been derived from a selected population of activated mesenchymal stem cells.

6. The pharmacologically active preparation of claim 5 wherein the extracellular vesicles are derived from activated mesenchymal stem cells that have an enhanced level of TSG-6.

7. The pharmacologically active preparation of claim 5 wherein the activated mesenchymal stem cells are human activated mesenchymal stem cells.

8. A method for providing a pharmacologically active preparation of selected mesenchymal stem cell derived extracellular vesicles, said method comprising:
culturing a population of mesenchymal stem cells in a serum free medium so as to provide an activated population of mesenchymal stem cells;
culturing the activated population of mesenchymal stem cells under conditions suitable for production of extracellular vesicles so as to provide a mesenchymal stem cell derived population of extracellular vesicles having pharmacological activity; and
isolating the mesenchymal stem cell derived extracellular vesicles to provide a pharmacologically active preparation enriched for mesenchymal stem cell derived extracellular vesicles,
wherein the pharmacologically active preparation of the extracellular vesicles possesses an enhanced anti-inflammatory activity.

9. The method of claim 8 wherein the population of mesenchymal stem cells are human mesenchymal stem cells.

10. A pharmaceutically acceptable preparation comprising a pharmacologically active preparation of extracellular vesicles derived from a population of activated mesenchymal stem cells, and a pharmaceutically acceptable carrier solution.

11. The pharmaceutically acceptable preparation of claim 10 wherein the pharmaceutically acceptable carrier solution is saline.

12. The pharmaceutically acceptable preparation of claim 10 wherein the activated mesenchymal stem cells express an enhanced level of TSG-6.

13. The pharmaceutically acceptable preparation of claim 10 wherein the mesenchymal stem cells are human mesenchymal stem cells.

14. A method for inhibiting onset of specific autoimmune disease in type 1 diabetes comprising administering the pharmaceutically acceptable preparation of claim 6 to an animal in need thereof.
